(19) United States
(12) Patent Application Publication
(10) Pub. No.: US 2007/0214521 A1
(43) Pub. Date: Sep. 13, 2007

(54) Role of microRNA in plant salt tolerance

(75) Inventors: Jian-Kang Zhu, Riverside, CA (US); Xiangyang Hu, Shanghai (CN); Jian-Hua Zhu, Riverside, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 11/371,395

(22) Filed: Mar. 8, 2006

(51) Int. Cl. A01H 1/00; C07H 21/04; C12N 15/82; C12N 5/04

(52) U.S. Cl. 800/289; 800/294; 435/419; 435/468; 536/23.6

(57) ABSTRACT

The present invention provides nucleic acid constructs encoding microRNA molecules that can be used to confer salt tolerance on plants. The invention further provides transgenic plants comprising the nucleic acids, as well as methods of using them to confer salt tolerance on plants.

### STATEMENT AS TO RIGHTS TO INVENTIONS MADE UNDER FEDERALLY SPONSORED RESEARCH AND DEVELOPMENT

This invention was made with Government support under Grant No. 0212346, awarded by the National Science Foundation. The Government has certain rights in this invention.

### FIELD OF THE INVENTION

This invention relates to control of plant gene expression in response to stress. In particular, it relates to microRNAs that are useful in down-regulating plant gene expression in response to soil salinity.

### BACKGROUND OF THE INVENTION

Plants have evolved sophisticated anatomical, physiological and molecular responses to environmental stresses such as soil salinity, extreme temperatures, and water deprivation (Frommer, et al., Science 285:1222 (1999); Hasegawa, et al., Annu. Rev. Plant Physiol. Plant Mol. Biol. 51:463 (2000); Bartels, Trends Plant Sci. 6:284 (2001), Zhu, Annual Review of Plant Biology 53:247 (2002); Apel, et al. Annual Review of Plant Biology 55:373 (2004) Seki, et al., Plant Cell 13:61 (2001) Fowler et al., Plant Cell 14:1675 (2002); Amtmann et al. Plant Physiol. 138:127 (2005)). Soil salinity is one of the most important abiotic stresses limiting agricultural productivity and disrupts plant function by a number of mechanisms (Hasegawa, et al., Annu. Rev. Plant Physiol. Plant Mol. Biol. 51:463 (2000); Amtmann et al. Plant Physiol. 138:127 (2005)). Salt, and other abiotic stresses, cause both up and down regulation of gene expression (Seki, et al., Plant Cell 13:61 (2001) Fowler et al., Plant Cell 14:1675 (2002); Amtmann et al. Plant Physiol. 138:127 (2005)). Many stress up-regulated genes encode proteins known or presumed to be required for stress tolerance (Apel, et al. Annual Review of Plant Biology 55:373 (2004)). In contrast, down regulation of gene expression by stress is relatively under studied. For many of these genes, down-regulation by stress may be simply a consequence of reduced growth or photosynthesis. Other down regulated genes may be important negative determinants of stress tolerance, and their identification is an important part of understanding salt tolerance mechanisms. Recent studies have now firmly established microRNAs (miRNAs) as key regulators of gene expression (Bartel, Cell 116:281 (2004); Carrington and Ambros, Science 301:336 (2003); Voinnet, Nat. Rev. Genet. 6:206 (2005); Willmann et al. Curr Opin Plant Biol. 8:548 (2005); Palatnik, et al, Nature 425:257 (2003); Mallory et al. Plant Cell 17:1360 (2005); Kidner et al. Curr Opin Plant Biol. 8:38 (2005)). The best studied plant miRNAs target mRNAs encoding transcription factors involved in development (Willmann et al. Curr Opin Plant Biol. 8:548 (2005); Palatnik, et al., Nature 425:257 (2003); Mallory et al. Plant Cell 17:1360 (2005); Kidner et al. Curr Opin Plant Biol. 8:38 (2005)). However, many other plant miRNAs have now been predicted to target mRNAs that encode proteins involved in a wide range of cellular processes such as proteolysis, metabolism and nutrient transport (Sunkar and Zhu Plant Cell 16: 2001 (2004); Jones-Rhoades and Bartel, Molecular Cell 14:787 (2004)). The physiological consequences of miRNA-mediated post transcriptional regulation of these genes have yet to be investigated.

### BRIEF SUMMARY OF THE INVENTION

This invention provides methods of conferring salt tolerance on a plant. The methods comprise introducing into the plant a nucleic acid molecule comprising a polynucleotide sequence encoding an miRNA molecule at least 90% identical to miR397 (SEQ ID NO: 1). In typical embodiments, the nucleic acid molecule comprises an expression cassette comprising a promoter operably linked to the polynucleotide sequence encoding the miRNA molecule. The promoter may be a constitutive or an inducible promoter.

The nucleic acid may be introduced into the plant using any of a number of well-known techniques such as using Agrobacterium or by a sexual cross.

The invention also provides isolated nucleic acid molecule comprising a polynucleotide sequence encoding the miRNA molecules of the invention. The invention further provides transgenic plants comprising the expression cassettes of the invention.

Soil salinity is a severe constraint for plant agriculture, and novel approaches to improving plant salt tolerance are needed to sustain agricultural productivity. We found that genes encoding laccase-like proteins (referred to collectively as LAC) and a regulatory subunit of casein kinase (CKB3) are down-regulated by salt stress in Arabidopsis. This down-regulation is caused by salt stress-induced transcriptional-upregulation of miR397, which directs cleavage of the LAC and CKB3 transcripts. Overexpression of miR397 in transgenic Arabidopsis enhances LAC and CKB3 transcript cleavage and increases plant salt tolerance, whereas overexpression of miR397-resistant forms of LAC and CKB3 reduces salt tolerance. These results demonstrate that miR397-guided down-regulation of LAC and CKB3 expression is essential for salt tolerance, and that manipulation of miRNA expression is an effective new approach to improving plant salt tolerance.

### DEFINITIONS

The terms “microRNA” or “miRNA” refer to short (about 18 to about 26 nucleotides), endogenous noncoding RNAs found in animals and plants. miRNAs can be identified by cloning and by computational approaches based on sequences conserved among known miRNAs (Dugas and Bartel. Curr. Opin. Plant Biol. 7:512-520 (2004)).

A “salt tolerant plant” of the invention is capable of growing under saline conditions which inhibit the growth of at least 95% of the parent, non-salt tolerant plant from which the salt tolerant plant is derived. Typically, the growth rate of salt tolerant plants of the invention will be inhibited by less than 50%, preferably less than 30%, and most preferably will have a growth rate which is not significantly inhibited by a growth medium containing water soluble inorganic salts which inhibits growth of at least 95% of the parental, non-salt tolerant plants.

In the case of plants, exemplary water-soluble inorganic salts commonly encountered in saline soils are alkali metal salts, alkaline earth metal salts, and mixtures of alkali metal salts and alkaline earth metal salts. These commonly include sodium sulfate, magnesium sulfate, calcium sulfate, sodium chloride, magnesium chloride, calcium chloride, potassium chloride and the like. Soil conductivity is typically used to determine the degree of salinity of a particular soil. Such soil conductivity measurement can be made in situ by standard procedures using a soil contacting Wenner Array four probe resistivity meter or other equivalent device.

The term “expression cassette” refers to any recombinant expression system for the purpose of expressing a nucleic acid sequence of the invention in vitro or in vivo, constitutively or inducibly, in any cell, including, in addition to plant cells, prokaryotic, yeast, fungal, insect or mammalian cells. The term includes linear or circular expression systems. The term includes all vectors. The cassettes can remain episomal or integrate into the host cell genome. The expression cassettes can have the ability to self-replicate or not, i.e., drive only transient expression in a cell. The term includes recombinant expression cassettes which contain only the minimum elements needed for transcription of the recombinant nucleic acid.

As used herein, the term “promoter” includes all sequences capable of driving transcription of a coding sequence in a plant cell. Thus, promoters used in the constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription. Promoters can be constitutive or inducible.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

Optimal alignment of sequences for comparison may be conducted by the local homology algorithm of Smith and Waterman Add. APL. Math. 2:482 (1981), by the homology alignment algorithm of Needle man and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 70% sequence identity, at least 80% sequence identity, preferably at least 85%, more preferably at least 90%, 93% and most preferably at least 95%, or 97% compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 70% sequence identity, at least 80% sequence identity, preferably at least 85%, more preferably at least 90%, 93% and most preferably at least 95%, or 97% compared to a reference sequence. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

Another indication that nucleotide sequences are substantially identical is if two molecules hybridize to each other, or a third nucleic acid, under stringent conditions. Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is about 0.02 molar at pH 7 and the temperature is at least about 60° C.

For the purposes of this disclosure, stringent conditions for hybridizations are those which include at least one wash in 0.2×SSC at 63° C. for 20 minutes, or equivalent conditions. Moderately stringent conditions include at least one wash (usually 2) in 0.2×SSC at a temperature of at least about 50° C., usually about 55° C., for 20 minutes, or equivalent conditions.

### BRIEF DESCRIPTION OF THE DRAWINGS

FIG. 1. Salt stress regulation of miR397, LAC and CKB3 A. Time course of miR397, LAC and CKB3 expression during salt stress. LAC At2g30210 does not contain a miR397 complimentary site. B. Promoter:GUS staining for miR397a and b, LAC and CKB3. Three-week-old transgenic seedlings were removed directly from control media or exposed to 200 mM NaCl for 24 h and stained for GUS activity.

FIG. 2. Manipulation of miR397 and its targets in transgenic plants. A. RNA blot analysis of miR397a (left panel) and m-miR397a (right panel) overexpressing plants. B. RNA blot analysis of miR397b-overexpressing plants. C. Sequences of miR397, m-miR397, mLAC and mCKB3 compared to the miR397 target site in wild type LAC and CKB3. The introduced point mutations used to disrupt miR397 complementarity in mLAC and mCKB are shown in red. D and E. RNA blot analysis of plants overexpressing LAC or mLAC (D) or CKB3 or mCKB3 (E). Left panels show samples collected from seedlings under control conditions for wild type and three transgenic lines. Right panels show samples collected at 0, 6 or 12 h after the start of 200 mM NaCl treatment for one overexpressing line.

FIG. 3. Overexpression of miR397 increases salt resistance. A. Phenotype of wild type and miR397a and b overexpressing seedlings after transfer of 7 day-old seedlings from control media to either fresh control media or media containing 200 mM NaCl. Photographs were taken 14 days after transfer. B. Growth of wild type and miR397a-overexpressing plants under either control or salt stress conditions. For salt treatment, 17-day-old plants were irrigated with 50 mM NaCl followed by 100 mM and 200 mM NaCl applied 5 and 9 days later respectively. Photographs were taken one week after application of 200 mM NaCl. All plants were 33-days-old at the time of photography.

FIG. 4. Overexpression of miR397 target genes increases salt sensitivity. A. Phenotype of wild type and LAC, mLAC, CKB3 or mCKB3 overexpressing transgenic seedlings after transfer of 7 day-old seedlings from control media to either fresh control media or media containing 100 mM NaCl. Photographs were taken 14 days after transfer. B. Growth of wild type and LAC or CKB3 overexpressing plants under either control or salt stress conditions. For salt treatment, 17-day-old plants were irrigated with 50 mM NaCl followed by 100 mM applied 5 days later. Photographs were taken 11 days after application of 100 mM NaCl. All plants were 33 days-old at the time of photography.

FIG. 5 miR397-directed cleavage of CKB3 mRNA as determined by RNA ligase-mediated 5′ RACE. The frequency of 5′RACE clones corresponding to the cleavage sites are indicated.

FIG. 6 A. Germination and growth of wild type and miR397a and b overexpressing seedlings on control media or media containing 100 mM or 200 mM NaCl. Pictures were taken after 10days of growth. B. Quantification of root elongation for seedlings treated as in A. Data are expressed as a percentage of the average root elongation on control media for each genotype. Data are means ±S.D. (n=5). Overexpression of miR397a or miR397b increased the growth and survival of salt stressed seedlings but did not affect growth of unstressed seedlings. miR397-overexpressing seedlings germinated and grown on salt-containing media had an 15% (100 mM NaCl) and 63% (200 mM NaCl) reduction in root growth while the wild type root elongation was reduced by 40% and 80% in the same two treatments. In agreement with the gene expression data, overexpression of either miR397a or miR397b had the same effect of increasing growth of salt stressed seedlings, again indicating the functional redundancy of these genes. C. Salt resistance phenotype of lac1 and ckb3 T-DNA knockouts. Photos were taken after 7 days of growth on control or 200 mM NaCl media. Seeds of the two T-DNA lines were obtained from the Arabidopis Biological Resource Center: Salk_{—}025690 contains an insertion in the fifth exon of the LAC At2g38080 (here designated as lac1) and Salk_{—}108997 contains an insertion in the first exon of CKB3. Homozygous plants of each line were isolated by PCR screening. No obvious growth or developmental phenotypes were observed for either line when grown under unstressed conditions. Under salt stress, lac1 and ckb3 seedlings had increased growth relative to wild type. Although the phenotype of lac1 and ckb3 was weaker compared to miR397 overexpressing plants (B), the results are consistent, and suggest that LAC and CKB3 are negative regulators of salt tolerance and down regulation of both LAC and CKB3 is required to have the maximal effect on salt tolerance.

FIG. 7 A. Germination and growth of wild type, LAC, mLAC, CKB3 or mCKB3 overexpressing seedlings on control media or media containing 100 mM or 200 mM NaCl. Pictures were taken after 7 days of growth. B. Quantification of root elongation for seedlings treated as in A. Data are expressed as a percentage of the average root elongation on control media for each genotype. Data are means ±S.D. (n=5). As could be hypothesized from the results from miR397-overexpressing plants, increased expression of LAC or CKB3 decreased seedling salt tolerance. When seedlings were germinated on media containing 100 mM NaCl, root length was decreased to around 50% of the control in seedlings overexpressing wild type LAC or CKB3 compared to 62% in wild type. However, in seedlings overexpressing miR397-resistant LAC or CKB3, root elongation was only 10% of the control when seedlings were grown on 100 mM NaCl.

FIG. 8 Salt resistance phenotype and LAC and CKB3 expression in wild type, miR397, m-miR397, LAC, mLAC, CKB3 and mCKB3 seedlings and F1 seedlings from crosses between the overexpression lines. Top panels show seedlings after 7 days of germination and growth on control media or media containing 100 mM or 200 mM NaCl. Lower panels show RNA blot analysis of unstressed seedlings. The blot was probed with full length cDNAs of LAC, CKB3 and a LAC not targeted by miR397 (At2g30210). rRNA staining is shown as a loading control. These crosses allowed us to further verify that it is the miR397-mediated cleavage of LAC or CKB3 mRNAs that leads to increased salt resistance by combining miR397 overexpression and target overexpression in the same plants and evaluating the effects on LAC and CKB3 transcript accumulation and salt resistance. Also, mLAC and mCKB3 plants were crossed to plants transformed with the mutated form of mir397 (m-miR397). The m-miR397 was designed to be complementary to the mutated target site in mLAC and mCKB3. Thus, crossing m-miR397 into a plant expressing mLAC or mCKB3 should restore cleavage of the transcript. Based on our other results, it would also be expected that this would restore the wild type level of salt tolerance. Overexpression of miR397 increased LAC and CKB3 transcript cleavage and seedling salt resistance (left side of figure). However, overexpression of m-miR397 which could not target LAC or CKB3 had no effect on seedling salt resistance. This again confirms that it is cleavage of the LAC or CKB3 transcripts, not miR397 overexpression itself, that affects salt resistance. Also in agreement with this point, overexpression of either wild type or mutated forms of LAC and CKB3 decreased seedling salt resistance (right side of figure). Crosses of miR397a or miR397b overexpression lines to wild type LAC or CKB3 had little accumulation of full length LAC transcript but a large accumulation of the LAC 3′-cleavage product and were salt resistant. This is consistent with the results of overexpressing miR397 by itself in that whenever LAC or CKB3 transcript is fully cleaved, seedling salt tolerance is increased. These crosses also demonstrated that that increased accumulation of the 3′ cleavage product of either LAC or CKB3 does not affect seedling growth or salt resistance. Crosses between plants miR397 overexpressing plants and plants overexpressing LAC or CKB3 transcripts mutated to abolish the miR397 complementary site were also of interest because they allowed us to examine the effect of high levels of full-length transcript of only one of the miR397 targets while the other miR397 targets were fully cleaved. For example, seedlings overexpressing miR397a or b and mLAC had high levels of full-length LAC transcript while CKB3 was completely cleaved (middle left of figure). These seedlings were salt sensitive. Likewise, seedlings overexpressing miR397a or b and mCKB3 had high levels of CKB3 full-length transcript but greatly reduced levels of LAC full-length transcript. These seedlings were also salt sensitive. Thus, high level of expression of only one of the miR397 target genes was sufficient to confer salt sensitivity. If LAC and CKB3 affected salt tolerance by unrelated mechanisms, we might have expected that overexpression of only one LAC or CKB3 but not the other would have only a partial effect on salt tolerance. The fact that either LAC or CKB3 overexpression alone was sufficient to confer a fully salt sensitive phenotype implies that LAC and CKB3 act as part of the same mechanism or very closely related mechanisms in determining salt stress resistance. Crosses of plants overexpressing mutated LAC or CKB3 with those overexpressing a mutated miR397 designed to restore cleavage of the mutated LAC or CKB3 transcripts (middle right) accumulated high levels of 3′ cleavage products but, still had wild type levels of the full length LAC or CKB3 transcript. This indicates that the mutated miR397 did lead to the cleavage of the mutated LAC or CKB transcripts and this did not affect accumulation of the wild type transcript. Consistent with this, the salt resistance phenotype of these seedlings was approximately the same as wild type.

FIG. 9 A. Soluble phenolic content of control and salt stressed seedlings assayed 24 h after transfer of 10 day-old seedlings to control, 100 mM NaCl or 200 mM NaCl media. Data are means ±SD (n=5). Although the function of LACs are largely unknown, they are know to be involved in metabolism of a range of cell wall lignins as well as soluble phenolics (Mayer and Staples, Phytochemistry 60:551 (2002)). Measurement of soluble phenolic content in plants with altered miR397, LAC or CKB3 expression allowed us to make an intial estimation of whether laccase acitivity is altered in these plants. None of the lines differed from wild type in phenolic content under unstressed conditions. Transfer of seedlings to 100 or 200 mM NaCl increased soluble phenolic content by 2- to 4-fold in wild type seedlings. A greater increase in salt-induced phenolic accumulation was seen in seedlings overexpressing miR397a or b, but not m-miR397 which cannot cleave LAC or CKB3. Conversely, overexpression of LAC or mLAC reduced the salt-induced phenolic accumulation to below wild type levels with mLAC overexpression having the greater effect.

Overexpression of CKB or mCKB had a similar effect, although to a lesser extent. Consistent with this, the lac1 and ckb3 T-DNA knockouts had higher than wild type accumulation of phenolics after salt treatment (data not shown) but the effect was smaller than that seen in the miR397 overexpression lines. Our results are in agreement with those of Ranocha, et al. Plant Physiol. 129:145 (2002) who found that antisense suppression of LAC expression could increase levels soluble phenolics. Thus, one reason that down regulation of LAC and CKB3 is required for salt resistance may be to allow the accumulation of soluble phenolics which may have a productive function under stress. B. Soluble peroxide content of control and salt stressed seedlings assayed 24 h after transfer of 10 day-old seedlings to control or 200 mM NaCl media. Data are means ±SD (n=5). To estimate the oxidative stress status of our transgenic lines under salt stress, we used the xylenol orange (FOX reagent) assay (Wolff, Methods Enzymol. 233:182 (1994)) to measure soluble peroxide content of control and salt stressed seedlings. After treatment with 200 mM NaCl, peroxide content of wild type seedlings increased two-fold. Peroxide content was slightly lower in miR397, but not m-miR397 overexpressing lines. The biggest difference, however was in the LAC and CKB3 overexpressing lines which had a four fold increase in peroxide content after salt treatment. For both LAC and CKB3, overexpression of a gene lacking the miR397 target site had a greater effect on peroxide content. This increase in peroxide content raises the possibility that the increased salt sensitivity of the LAC and CKB3 overexpressing lines is caused at least in part by increased oxidative damage. It is also possible that the reduced levels of soluble phenolics in LAC and CKB3 overexpressing plants (A) decreases their ROS scavenging capacity and leads to greater ROS build up and ROS-induced damage.

FIG. 10 In-gel MAPK assay of samples collected at the indicated times after transfer to 200 mM NaCl. MAPK activity is known to be induced by various abiotic stresses including cold and dehydration and activation of MAPK signaling has been implicated in stress tolerance (Jonak, et al. Proc. Natl. Acad. Sci. USA 93:11274 (1996); Kiegerl et al. Plant Cell 12:2247 (2000).). Also, MAPK signaling is known to be involved in oxidative stress (Kovtun et al. Proc. Natl. Acad. Sci. USA 97:2940 (2000)). We performed in-gel kinase assays to determine the relative activation of MAPK phosphorylation activity by salt stress in our wild type and transgenic seedlings. Wild type seedlings had elevated MAPK activity after 5, 15 or 30 min exposure to 200 mM NaCl. This MAPK activation was unaffected by overexpression of m-miR397 which cannot cleave LAC or CKB3. In seedlings overexpressing miR397a or b, however, MAPK activity was increased above the wild type level at all time points observed after salt treatment. In contrast, seedlings overexpressing LAC, CKB3, mLAC or mCKB3 had below wild type levels of MAPK activity after salt treatment. Thus, the extent to which MAPK activity was inversely correlated with both LAC and CKB3 expression and directly correlated with the relative resistance of seedling growth to salt stress.

### DETAILED DESCRIPTION

The present invention is based, at least in part, on the discovery that transgenic plants expressing miRNA molecules is an effective new approach to improving plant salt tolerance. In particular, the present invention provides evidence that expression of miR397 (UGUAGUUGCUACGUGAGUUACU, SEQ ID NO: 1) can down-regulate expression of genes and confer salt tolerance on the plants.

DNA constructs of the invention may be introduced into the genome of the desired plant host by a variety of conventional techniques. For example, the DNA construct may be introduced directly into the genomic DNA of the plant cell using techniques such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Agrobacterium tumefaciens—mediated transformation techniques, including disarming and use of binary vectors, are well described in the scientific literature. See, for example Horsch et al. Science 233:496-498 (1984), and Fraley et al. Proc. Natl. Acad. Sci. USA 80:4803 (1983).

Transformed plant cells which are derived by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker which has been introduced together with the desired nucleotide sequences.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

To use isolated sequences in the above techniques, recombinant DNA vectors suitable for transformation of plant cells are prepared. Techniques for transforming a wide variety of higher plant species are well known and described in the technical and scientific literature. The vectors of the invention will typically comprise an expression cassette comprising the DNA sequence coding for the desired miRNA combined with transcriptional and translational initiation regulatory sequences which will direct transcription in the intended tissues of the transformed plant.

For example, a plant promoter fragment may be employed which will direct expression of the miRNA in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, and other transcription initiation regions from various plant genes known to those of skill.

Alternatively, the plant promoter may direct expression of the desired miRNA in a specific tissue or may be otherwise under more precise environmental or developmental control. Examples of environmental conditions that may effect transcription by inducible promoters include anaerobic conditions, elevated temperature, or the presence of light. Such promoters are referred to here as “inducible” or “tissue-specific” promoters. One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

The vector comprising the expression cassettes of the invention will typically comprise a marker gene which confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosulfuron or Basta.

The expression cassettes of the invention can be used to confer a desired trait on essentially any plant. Thus, the invention has use over a broad range of plants, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and, Zea.

### EXAMPLES

The following examples are offered to illustrate, but not to limit the claimed invention.

### Materials and Methods

Plant Material and Salt Stress Tolerance Assays

Arabidopsis thaliana Ecotype Columbia was used as the wild type and is the genetic background for all mutants and transgenic plants used in this study. For agar-plate based assays of seedling growth or salt tolerance, seeds were surface-sterilized and sown plates containing on MS nutrient media with 3% sucrose and 0.6% agar. Seeds were stratified at 4° C. for 3 d and then transferred to 22° C. under continuous light for measurements of germination and growth. For salt tolerance assays, seedlings were either germinated directly on salt-containing media or transferred to salt-containing media after 5 days of growth on control media. Seedling survival was scored as the number of seedlings having green cotyledons and leaves. Root growth was quantified by marking the position of the root apex on the back of the plate. NaCl concentrations used and length of salt exposure are indicated in the text or figure legends describing each experiment.

RNA Isolation and Analysis

Total RNA was isolated using Trizol Reagent (Invitrogen). For analysis of high molecular weight RNA, thirty micrograms of total RNA was fractionated on a 1% agarose gel containing formaldehyde and then blotted onto a nylon membrane (HYBond N+, Amersham Biosciences). 32P-labeled probes were generated with Ready-to-go DNA labeling kit (Amersham) from either full length or a 5′ fragment of the LAC or CKB3 open reading frame. The 5′ probe of LAC consisted of 600 bp downstream of the start codon of the LAC (At2g38080) ORF , the 5′ probe of CKB3 consisted of the 100 bp including the 50 bp of upstream of start codon and 50 bp downstream of the start codon of CKB3 cDNA. Membranes were hybridized in 50% formamide, 5×SSC, 25mM sodium phosphate buffer (pH 6.5),10× Denhardt's solution, and 250 μg/ml denatured salmon sperm DNA at 42° C.; washed once with 2×SSC, 0.1% SDS and once more with 0.1× SSC, 0.1% SDS at 68° C., briefly air dried and exposed to X-ray films (Mallory et al. Plant Cell 17:1360 (2005)). Each lane contained 20 μg (miRNA analysis) or 30 μg (LAC and CKB3 analysis) of total RNA isolated from 15-day-old wild-type seedlings after exposure to 200 mM NaCl for the times indicated. For miR397 analysis, blot was probed with 32P-labeled DNA complementary sequence to miR397 and with sequence complementary to miR171 as a loading control. For LAC or CKB3 analysis, the membrane was hybridized with 32P-labeled fill length or 5′ specific fragments of LAC or CKB3 as indicated, or with a LAC (At2g30210) or CKB2 (At4g17640) not targeted by miR397 (as a loading control and to show lack of cleavage of this gene). For analysis of small RNAs, twenty micrograms of total RNA was separated on a denaturing 15% polyacrylamide gel, and transferred electrophoretically to Hybond-N+ membranes (Amersham Bioscience, Buckinghamshire, UK). Hybridization and washings were performed as previously described (Sunkar and Zhu, Plant Cell 16:2001 (2004)). For small RNA blots, 20 μg of total RNA was loaded and the blot probed with DNA probes complementary to miR397 or m-miR397a. Blots were reprobed with U6 RNA (left panel) or miR171 (right panel) as a loading control.

5′ RACE Analysis of mRNA Cleavage

Total RNA was extracted from seedlings using Trizol reagent and Poly(A)+ mRNA purified using a Poly A purification kit (Promega). RNA ligase-mediated 5′ RACE was performed using the GeneRacer kit (Invitrogen). The GeneRacer RNA Oligo adapter was directly ligated to mRNA (100 ng) without calf intestinal phosphatase and tobacco acid pyrophosphatase treatment. Initial PCR was performed with the GeneRacer 5′ primer and gene-specific primers for CKB3. Nested PCR was performed with 1 μL of the initial PCR reaction, the GeneRacer 5′ nested primer, and a CKB3 gene-specific internal primer. After the second amplification, PCR products were gel purified and cloned and 10 independent clones sequenced.

Overexpression and Promoter Fusion Constructs and Transgenic Plant Generation

To generate 35S:miR397a and 35S:miR397b constructs, a 300 bp fragment surrounding the miRNA sequence that includes the foldback structure of miR397a or miR397b was amplified from genomic DNA using primers indicated in the Supplemental data (Table 1). The amplified fragments were digested and cloned into XbaI and KpnI sites of pCAMBIA2305 downstream of the CaMV 35S promoter. To introduce point mutations into the miR397a precursor, PCR was performed using miR397a containing pCAMBIA2305 plasmid as template using the mutagenic primers indicated in Table 1. The first-round PCR products were gel-purified and used as template for second amplification and the resulting product was digested and cloned into the pCAMBIA2305. This fragment was sequenced to ensure that only the desired mutations were introduced.

To generate 35S:LAC and 35S:CKB3 constructs, the LAC (At2g38080) or CKB3 ORF was amplified by RT-PCR using the indicated primers (Table 1). The PCR products were first cloned into pBluescript and verified by sequencing. Then, the LAC or CKB3 ORF was released by digesting with XbaI and KpnI and subcloned into pCAMBIA2305. To generate miR397-resistant version of LAC (mLAC) and CKB3 (mCKB3), mutagenic primers (Table 1) were used. The first-round PCR products were purified and used as template for second amplification and the resulting product was digested and cloned into the pCAMBIA2305 and the clone verified by sequencing.

For miR397a and b, miR397b, LAC (At2g38080) and CKB3 promoter:GUS constructs, 2.0 kb fragments upstream the transcriptional start site were amplified using the indicated primer pairs (Table 1). The amplified products were digested with XbaI and BamHI and cloned into pBI101 plasmid.

All the constructs described were electroporated into Agrobacterium tumifaciens GV3101, which was used to transform Arabidopsis thaliana (ecotype Col-gl) using the floral dip method (Clough and Bent, Plant J. 16:735 (1998)). T3 homozygous lines were used for all experiments presented.

GUS Staining and Quantification of Soluble Phenolics and Peroxides

Histochemical localization of GUS activities in the transgenic seedlings or different tissues were analyzed after incubating the transgenic plants overnight at 37° C. in 1 mg/mL 5-bromo-4-chloro-3-indolyl-β-glucuronic acid, 5 mM potassium ferricyanide, 5 mM potassium ferrocyanide, 0.03% Triton X-100, and 0.1 M sodium phosphate buffer at pH 7.0. Soluble peroxide content was assayed using the method of ferrous ion oxidation in the presence of xylenol orange essentially as described by (Wolff, Methods Enzymol. 233:182 (1994)). Briefly, seedlings were ground in liquid nitrogen and suspended in extraction buffer (100 mM Tris-Cl pH 7.5, 5 mM EDTA, 10 mM MgC, 2). After centrifugation and filtration to remove insoluble material, aliquots of the extract were added to the assay solution (0.25 mM FeSO4, 0.25 mM (NH4)2SO4, 25 mM H2SO4, 1.25 μM xylenol orange, 1 mM sorbitol); absorbance at 560 nm quantified and peroxide content calculated by comparison to H2O2 standards.

For quantification of soluble phenolics, seedling tissue was extracted three times using 50% methanol containing 1% HCl and the extracts combined and evaporated to dryness. Samples were resuspended in water, filtered and phenolic content assayed by addition of Folin-Cioucalteu reagent (Sigma) and reading absorbance at 675 nm after stopping the reaction by addition of sodium carbonate. Soluble phenolic content was calculated by comparison to a standard curve prepared using gallic acid.

In-Gel MAPK Assay

In-gel MAPK assay was performed as described (Desikan et al. Exp. Bot. 50:1863 (1999)). Briefly, total protein was extracted from seedlings and 40 μg separated by SDS-PAGE using 10% gels containing 0.5 mg ml-1 bovine myelin basic protein (Sigrna). After washing and renaturation, the gel was incubated with reaction buffer containing 50 mM ATP and 50 mCi γ-32P ATP (PerkinElmer) for 1 h, washed to remove unincorporated radioactivity and exposed to autoradiography film.

Results

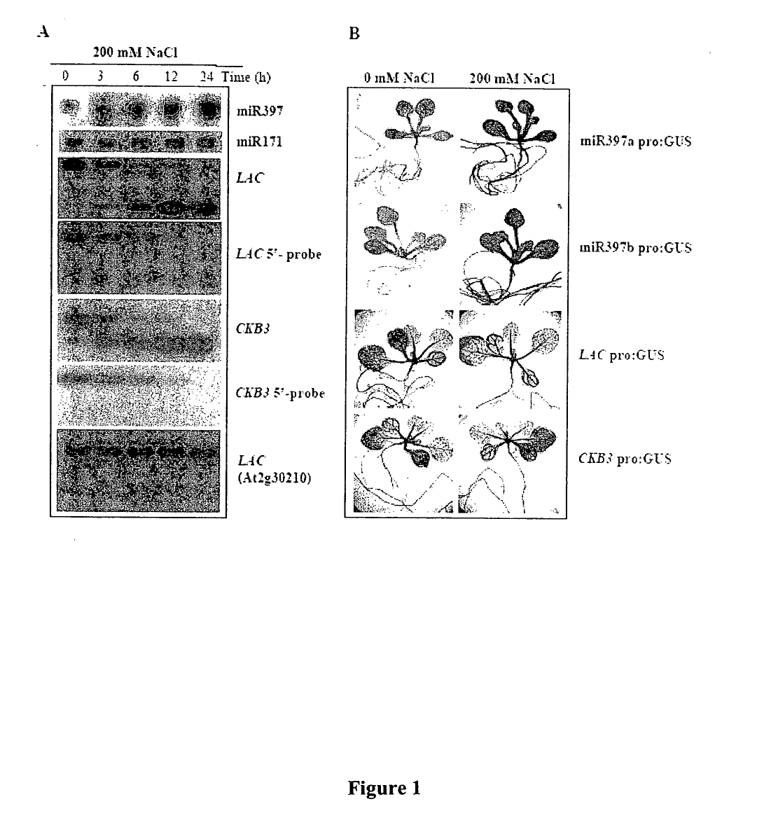

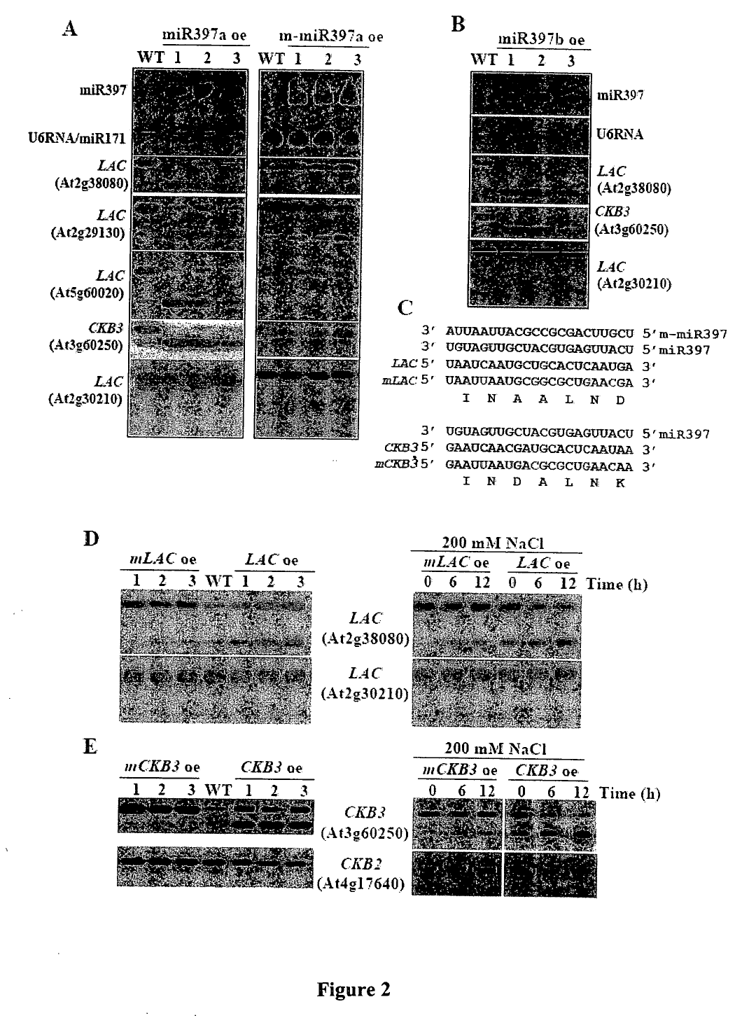

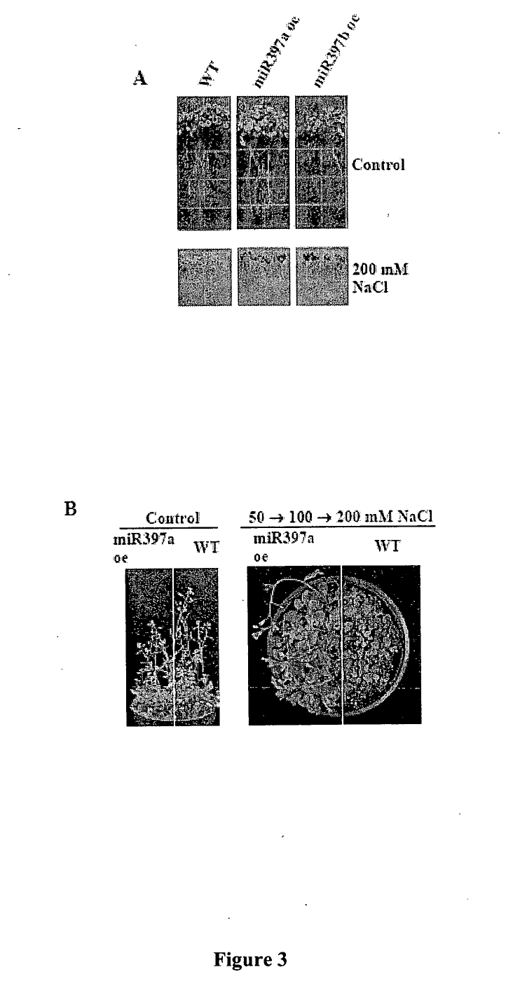

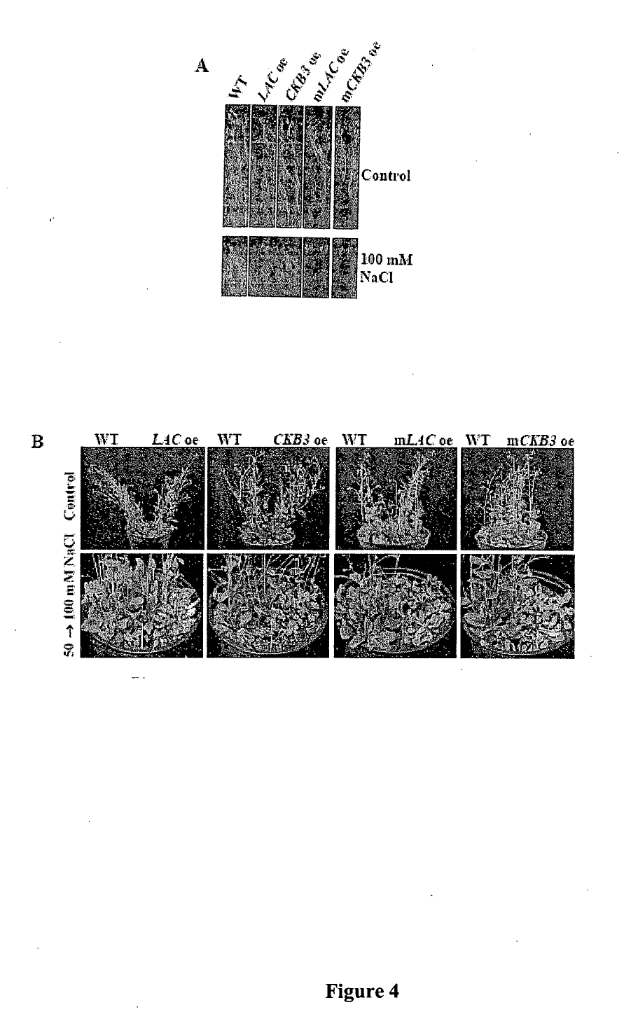

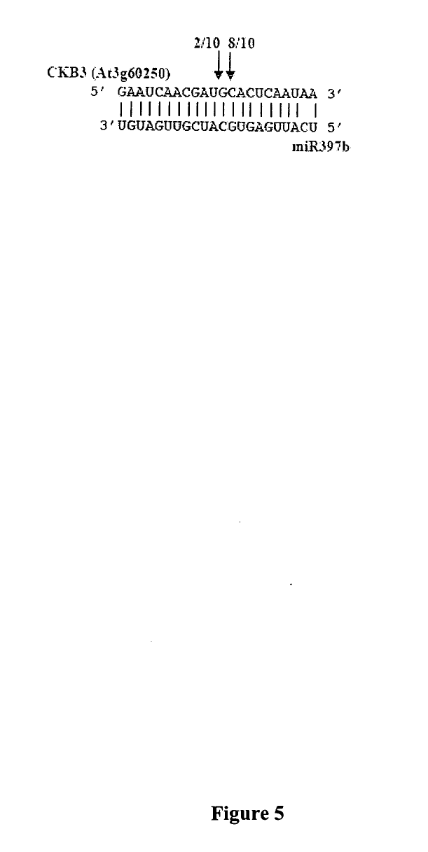

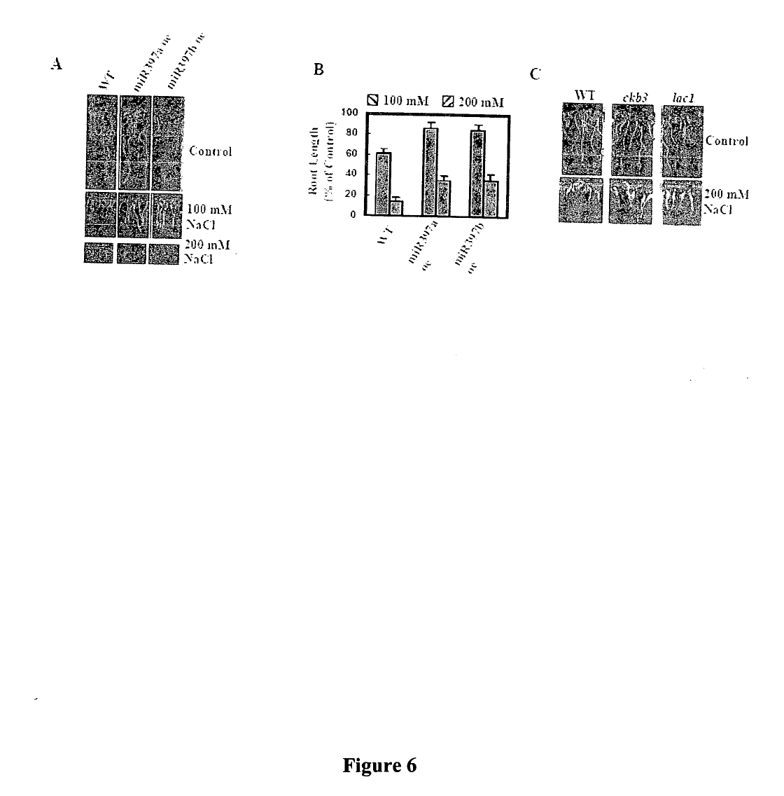

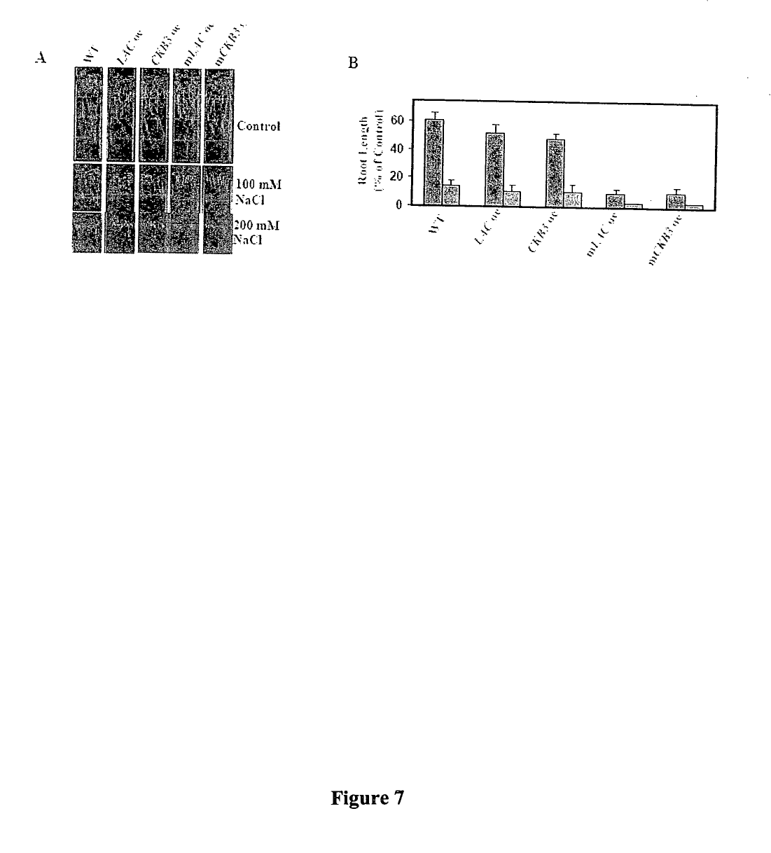

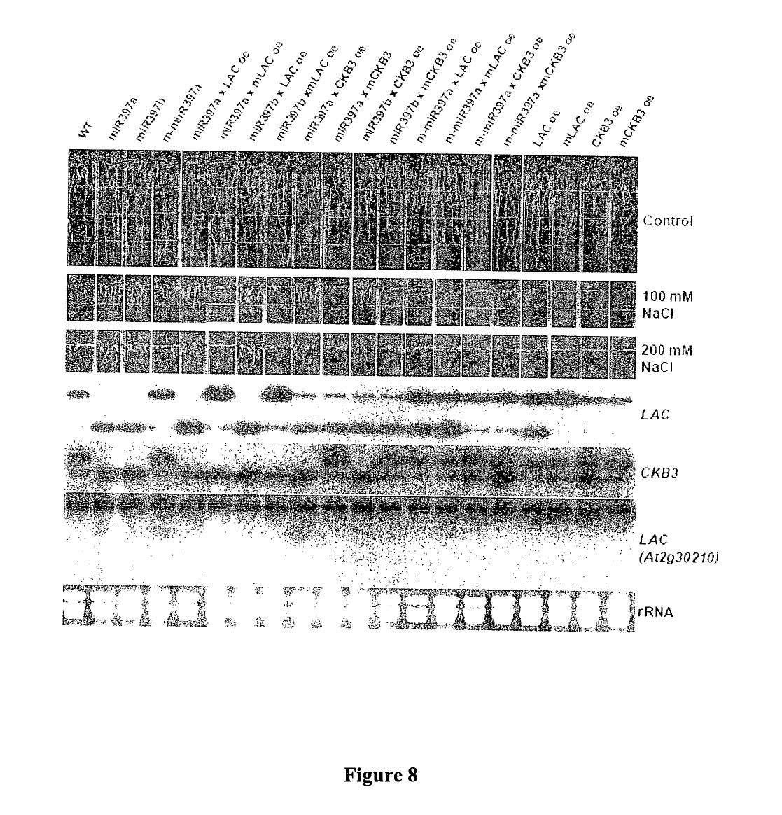

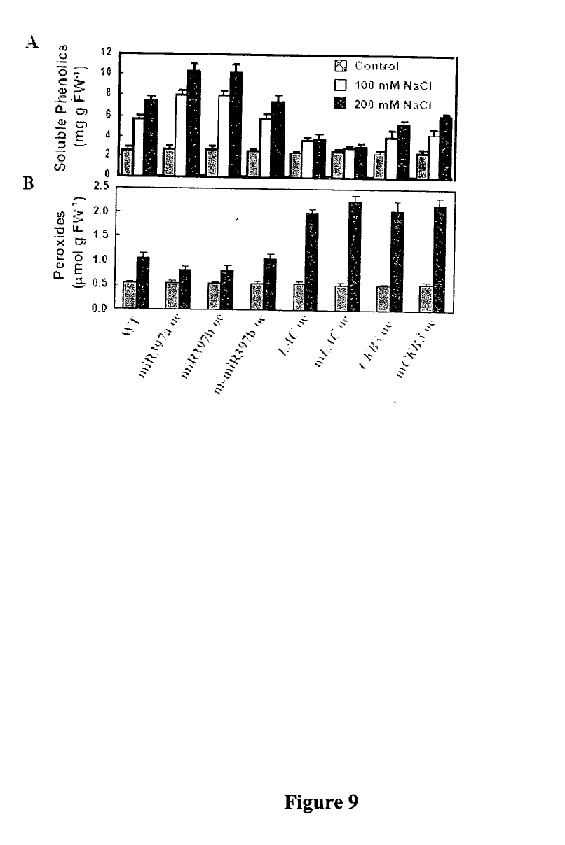

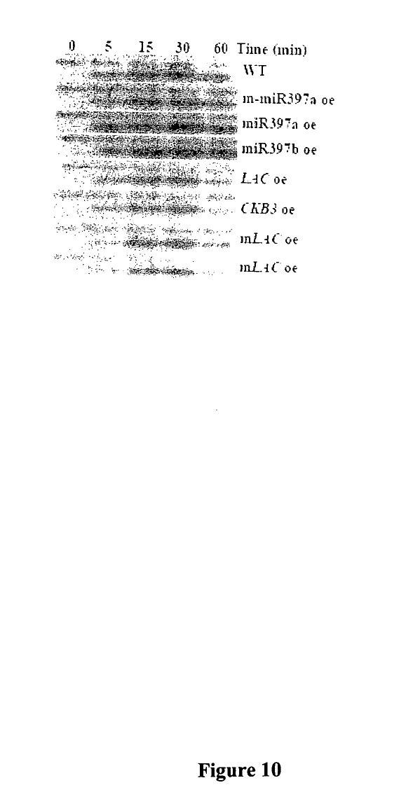

We found a dramatic upregulation of Arabidopsis miR397 by salt stress (FIG. 1A). miR397 is a conserved miRNA (Sunkar, and Zhu, Plant Cell 16:2001 (2004); Jones-Rhoades and Bartel, Molecular Cell 14:787 (2004)) with four predicted targets in Arabidopsis: three closely related genes encoding laccase-like proteins (LACs: At2g29130, At2g38080 and At5g60020) and casein kinase β subunit 3 (CKB3; At3g60250). 5′ RACE assays confirmed miR397 directed cleavage of mRNAs of the LACs (Jones-Rhoades and Bartel, Molecular Cell 14:787 (2004)) and CKB3 (FIG. 5). These miR397 targets showed a reduction in full length transcript and concomitant increase in putative 3′ cleavage product in salt treated plants (FIG. 1A; essentially identical results are also seen for the other two LACs). In contrast, expression of a LAC 1 not targeted by miR397 was not affected by salt treatment. miR397 is encoded by two genes which produce miRNAs differing in only one nucleotide (named miR397a and miR397b respectively) (Llave, et al., Science 297:2053 (2002)). Promoter:GUS analysis indicates that both miR397a and miR397b are transcriptionally upregulated by salt stress (FIG. 1B). In contrast, transcriptional upregulation of LAC (At2g38080) and CKB3 could not be detected; consistent with a post transcriptional down regulation of these transcripts in salt stressed plants. Transgenic plants ectopically overexpressing miR397a (or miR397b) but not a mutated form of miR397a (m-miR397), which does not complement the LAC or CKB3 transcripts, had decreased levels of LAC and CKB3 full length transcripts and increased levels of 3′ cleavage products (FIGS. 2A and B). Microarray analysis of both miR397a and miR397b overexpressing plants confirmed these results and showed that expression of other LACs and CKBs lacking a miR397 complementary site was not affected by miR397 overexpression (data not shown). We also performed the converse experiments of overexpressing either wild type LAC or CKB3 transcripts targeted by miR397 and mutated versions of the same transcripts where the miR397 target site was abolished. When designing the miR397-resistant forms of the targets, the corresponding amino acid sequence was unaltered and the mutated sequences match that of m-miR397 (FIG. 2C). In plants overexpressing the wild type version of a miR397-targeted LAC, there was only a modest increase in the amount of full length transcript while the amount of the 3′ cleavage product was dramatically increased (FIG. 2D). In contrast, transgenic plants overexpressing the mutated LAC (mLAC) had substantially increased amounts of full-length LAC transcript but no change in abundance of the 3′ cleavage product. This demonstrates that abolishing the miR397-complementary site in LAC also abolished cleavage of the transcript and allowed much higher accumulation of the full length mRNA. Abolishing the miR397-complementary site also abolished the salt stress-induced down regulation of LAC (FIG. 2D); demonstrating that miR397-mediated MRNA cleavage is required for salt stress down regulation of this gene. Essentially identical results were seen with plants overexpressing CKB3 or mCKB3 (FIG. 2E). Overexpression of miR397a or miR397b increased seedling survival after transfer of seedlings from control media to 200 mM NaCl media (FIG. 3A); more than 95% of miR397 overexpressing seedlings survived transfer to 200 mM NaCl whereas only approximately 10% of wild type seedlings survived. miR397-overexpressing seedlings also had enhanced root growth in less severe salt stress treatments (FIGS. 6, A and B). Soil grown plants progressively irrigated with 50, 100 and 200 mM NaCl grew substantially more than wild type (FIG. 3B). Growth of the wild type and transgenic plants was similar in the absence of salt stress. Essentially identical results were obtained with plants overexpressing miR397b (data not shown). We hypothesized that T-DNA knock-out mutants of LAC and CKB3 may also be more salt resistant. This was found to be true (FIG. 6C) but the effect was less dramatic than with miR397 overexpression. The combined data show that the coordinated down regulation of LAC and CKB3 by miR397 is a key determinant of salt tolerance. Conversely, transgenic plants overexpressing either wild type LAC or CKB3 or miR397-resistant mLAC or mCKB3 became more salt sensitive. Consistent with their greater accumulation of full length transcript, the greatest effect was seen in mLAC and mCKB3 overexpressing plants which could not survive transfer to even a relatively low level of salt (100 mM, FIG. 4A). Root growth of LAC and CKB3 overexpression plants was also more sensitive to salt than wild type (FIG. 7). In soil, wild type plants irrigated with 50 mM NaCl for 5 days and then 100 mM NaCl for 11 days showed minimal damage. In contrast, plants overexpressing either wild type or mutated LAC or CKB3 were more damaged by the salt treatment and grew less than wild type (FIG. 4B). Again, plants overexpressing miR397-resistant LAC or CKB3 had the greatest salt sensitivity; most likely because of their greater accumulation of full length LAC or CKB3 transcripts. The combined results suggest a strong negative relationship between the level of LAC and CKB3 expression and salt tolerance. We performed pair wise crosses between miR397 or m-miR397a overexpressing plants and plants overexpressing LAC, CKB3, mLAC or mCKB3 (FIG. 8). The results showed that overexpression of miR397 could abolish the effect of overexpressing LAC or CKB3, but not mLAC or mCKB3, on full-length transcript accumulation and salt tolerance. In contrast, overexpression of m-miR397 could abolish the effect of overexpressing mLAC or mCKB3 but not LAC or CKB3. Therefore, these results demonstrate that it is the miR397-mediated cleavage of LAC and CKB3 mRNAs that leads to increased salt resistance. In addition to a new role for miRNA-mediated post transcriptional regulation, this study has identified four new genes as negative determinants of salt stress resistance. Both of the gene families identified, LACs and casein kinases, are enigmatic in terms of their molecular and physiological functions. LACs, p-diphenol:dioxygen oxidoreductases, are a diverse family of enzymes present across higher plants and fungi and can oxidize a wide range of phenolic compounds (Mayer and Staples, Phytochem. 60:551 (2002)). LACs can produce reactive oxygen species in the presence of a suitable substrate (Mayer and Staples, Phytochem. 60:551 (2002)). Thus, one possible mechanism by which LAC could affect salt stress response is by altering ROS production or signaling. Consistent with this, overexpression of LAC or CKB3 increased the peroxide content of salt stressed seedlings while overexpression of miR397 decreased peroxide content (FIG. 9B). Also, laccases can function in the oxidative degradation of phenolic compounds (Kiegerl, et al., Plant Cell 12, 2247 (2000)), and it has been proposed that phenolic acid and related compounds are responsible for buffering the damaging effects of ROS (Tamagnone, et al., Plant Cell 10, 1801 (1998)). Consistent with this, we found that LAC and mLAC overexpressing plants have reduced levels of soluble phenolics under salt stress while overexpression of miR397 increased phenolic content (FIG. 9A). In CKB3 and mCKB3 overexpressing lines, there was also a slight decrease in soluble phenolics. This result may imply that CKB3 regulates LAC enzyme activities posttranslationally, since CKB3 does not appear to regulate LAC transcript levels (FIG. 8). In addition, LACs have been observed to have ferroxidase activity (Hoopes, et al. Plant Physiol. Biochem. 42:27 (2004)) and this could also be a mechanism by which LACs affect ROS production and salt tolerance. Unlike LAC, CKB3 has a well established role in signaling. The CK2 holoenzyme is composed of two a and two β subunits. Although traditionally viewed only as a component of tetrameric CKII complexes, CKIIβ may have functions independent of its role as the regulatory subunit of CKII (Bibby and Litchfield, Int J Biol Sci. 1:67 (2005)). In animals, CKIIβ inhibits MAPK activation mediated by c-Mos, a germ cell-specific serine/threonine protein kinase (Chen, et al., Mol. Cell Biol. 17:1904 (1997)). MAPK signaling has been implicated in various plant stress responses (Kiegerl, et al., Plant Cell 12:2247 (2000); Shou, et al., Proc. Natl. Acad. Sci. U.S.A. 101, 3298 (2004); Kovtun, et al., Proc. Natl. Acad. Sci. U.S.A. 97, 2940 (2000); Rentel, Nature 427, 858 (2004)). We found that CKB3 overexpression had a repressive effect on MAPK activation by salt stress (FIG. 10). MAPK activation may be beneficial for salt tolerance in part by inhibiting H2O2 accumulation (Moon, et al., Proc. Natl. Acad. Sci. U.S.A. 100:358 (2003)). CK2 has been shown to have a range of protein substrates including the stress protein RAB17 in maize (Riera, Proc. Natl. Acad. Sci. USA 101:9879 (2004)). Determining which of these targets is actually relevant to the negative role of CKB3 on salt tolerance will be of interest in future studies. CKB3 is not related to LAC in terms of structure or biochemical function but our results suggest that these genes are functionally related in being negative determinants of salt stress response. Also, knockout or overexpression of LAC or CKB3 while leaving the other unaffected is sufficient to alter salt tolerance in a manner similar to that seen when expression of both LAC and CKB3 is altered. Although it remains unknown how LAC and CKB3 affect salt tolerance, these results suggest they are part of a single mechanism. Thus, miR397 is an example of a miRNA whose targets are functionally related rather than being part of a single gene family.

In summary, our identification of a salt stress induced miRNA and demonstration of its critical role in salt tolerance establishes miRNA-mediated post transcriptional regulation as an integral component of salt stress response. The identification here of two seemingly disparate classes of genes, LACs and CKB3, that are linked through their regulation by miR397 also sets the stage for further characterization of the action of these genes in salt stress response and how they might also be functionally linked. Importantly, our results show that manipulation of miRNA expression is an effective new approach to improving plant salt tolerance.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

TABLE 1
Oligonucleotides used in this study.

| | |
|---|---|
| Oligonucleotide (5′-3′) | Oligo name |

Cloning miR397a and b

| | |
|---|---|
| GCTCTAGAGCGTACATTACAAACACTTGGAC | miR397a-F |
| GGGGTACCCCAGATTGATTACGTAAAGAACC | miR397a-R |
| GCTCTAGAGCAATAGTCACGCTACCTTTAGG | miR397b-F |
| GGGGTACCCCTTAGTAGATACGAAAACATGC | miR397b-R |
| miR397a mutagenesis | |
| TCGTTCAGAGCCGCATTAATGTAAATTCTTTTAATCC | miR397a-MR |
| ATTCAACAATG | |
| TCGTTCAGAGCCGCATTAATGTAATTTCGTTTTGTTT | miR397a-MF |
| TTCATTGTTAATGGA | |
| Cloning LAC and CKB3 | |
| GCTCTAGAGCATGGGGTCTCATATGGTTT | LAC-F |
| GGGGTACCCCTTAGCACAAGGGAA | LAC-R |
| GCTCTAGAGCATGTACAAGGAACG | CKB3 |
| GGGGTACCCCTCATGGTTTGTGTACC | CKB3 |
| LAC and CKB3 mutagenesis | |
| TTAACGCGGCGCTGAACGAAGAACTCTTTTTC AAAG | LAC-MF |
| TCGCCGGC | |
| TCGTTCAGCGCCGCGTTAACTAGTCGTAGCAG ATA | LAC-MR |
| GG | |
| ATTAATGACGCGCTGAATCAAGAAACTAGAG | CKB3-MF |
| AAATCTTCAACTTCCACC | |
| GATTCAGCGCGTCATTAATTCGTTTCCGATCAA TAG | CKB3-MR |
| CTCC | |
| Cloning the miR397a and b promoters | |
| GCTCTAGAGCTTGCGCTTCGTACCGGTGAGCG | miR397a-GUS.F |
| CGGGATCCCGCCAGGAAAAAATATCCTCATGC | miR397a-GUS.R |
| GCTCTAGAGCTCAATGATGTTCATTCAAACCC | miR397b-GUS.F |
| CGGGATCCCGTTTGGAAGTTTTGGGTTTCTCC | miR397a-GUS.R |
| Cloning LAC and CKB3 promoters | |
| AAA TTA CAA AAC CAA GAG ATC CAC GAC G | LAC-GUS.F |
| CGGGATCCCTCCCTCTCTATCTTTCTCTTCTCTCTC | LAC-GUS.R |
| CGGGTCGACATAAAACAAAACATCGAATCCG | CKB3-GUS.F |
| CGGGATCCATTCGATTCCTTCTCCAAAAAGAC | CKB3-GUS.R |
| CKB3 5′ RACE | |
| ACC ACA AAG ATT GAA ATC ATC TTG | CKB3-RACE1 |
| AAA TTA CAA AAC CAA GAG ATC CAC GAC G | CKB3-RACE2 |

What is claimed is:

1. A method of conferring salt tolerance on a plant, the method comprising introducing into the plant a nucleic acid molecule comprising a polynucleotide sequence encoding an miRNA molecule at least 90% identical to miR397 (SEQ ID NO: 1).

2. The method of claim 1, wherein the miRNA molecule is SEQ ID NO: 1.

3. The method of claim 1, wherein the nucleic acid molecule comprises an expression cassette comprising a promoter operably linked to the polynucleotide sequence encoding the RNA molecule at least 90% identical to SEQ ID NO: 1.

4. The method of claim 3, wherein the promoter is a constitutive promoter.

5. The method of claim 3, wherein the promoter is an inducible promoter.

6. The method of claim 1, wherein the nucleic acid is introduced into the plant using Agrobacterium.

7. The method of claim 1, wherein the nucleic acid is introduced into the plant by a sexual cross.

8. An isolated nucleic acid molecule comprising a polynucleotide sequence encoding an miRNA molecule at least 90% identical to miR397 (SEQ ID NO: 1).

9. The isolated nucleic acid molecule of claim 8, wherein the miRNA molecule is SEQ ID NO: 1.

10. The isolated nucleic acid molecule of claim 8, which comprises an expression cassette comprising a promoter operably linked to the polynucleotide sequence encoding the RNA molecule at least 90% identical to SEQ ID NO: 1.

11. The isolated nucleic acid molecule of claim 10, wherein the promoter is a constitutive promoter.

12. The isolated nucleic acid molecule of claim 10, wherein the promoter is an inducible promoter.

13. A transgenic plant comprising an expression cassette comprising a promoter operably linked to the polynucleotide sequence encoding the RNA molecule at least 90% identical to SEQ ID NO: 1.

14. The transgenic plant of claim 13, wherein the miRNA molecule is SEQ ID NO: 1.


SEQUENCE LISTING (32 sequences)

SEQ ID NO: 1 - RNA, 22 nt, Artificial Sequence
  microRNA miR397, miR397b
ucauugagugcaucguugaugu

SEQ ID NO: 2 - RNA, 22 nt, Artificial Sequence
  mutated form of miR397a (m-miR397)
ucguucagcgccgcauuaauua

SEQ ID NO: 3 - RNA, 22 nt, Artificial Sequence
  wild type laccase-like protein (LAC) transcript miR397 target site
uaaucaaugcugcacucaauga

SEQ ID NO: 4 - RNA, 22 nt, Artificial Sequence
  mutated version of laccase-like protein (LAC) transcript where miR397 target site abolished
uaauuaaugcggcgcugaacga

SEQ ID NO: 5 - protein, 7 aa, Artificial Sequence
  laccase-like protein (LAC) transcript  miR397 target site corresponding amino acid sequence
INAALND

SEQ ID NO: 6 - RNA, 22 nt, Artificial Sequence
  wild type casein kinase beta regulatory subunit 3 (CKB3) transcript miR397 target site
gaaucaacgaugcacucaauaa

SEQ ID NO: 7 - RNA, 22 nt, Artificial Sequence
  mutated version of casein kinase beta regulatory subunit 3 (CKB3) transcript where miR397 target site abolished
gaauuaaugacgcgcugaacaa

SEQ ID NO: 8 - protein, 7 aa, Artificial Sequence
  casein kinase beta regulatory subunit 3 (CKB3) transcript miR397 target site corresponding amino acid sequence
INDALNK

SEQ ID NO: 9 - DNA, 31 bp, Artificial Sequence
  amplification RT-PCR miR397a cloning oligonucleotide primer miR397a-F
gctctagagcgtacattacaaacacttggac

SEQ ID NO: 10 - DNA, 31 bp, Artificial Sequence
  amplification RT-PCR miR397a cloning oligonucleotide primer miR397a-R
ggggtaccccagattgattacgtaaagaacc

SEQ ID NO: 11 - DNA, 31 bp, Artificial Sequence
  amplification RT-PCR miR397b cloning oligonucleotide primer miR397b-F
gctctagagcaatagtcacgctacctttagg

SEQ ID NO: 12 - DNA, 31 bp, Artificial Sequence
  amplification RT-PCR miR397b cloning oligonucleotide primer miR397b-R
ggggtaccccttagtagatacgaaaacatgc

SEQ ID NO: 13 - DNA, 48 bp, Artificial Sequence
  amplification PCR miR397a mutagenic oligonucleotide primer miR397a-MR
tcgttcagagccgcattaatgtaaattcttttaatccattcaacaatg

SEQ ID NO: 14 - DNA, 52 bp, Artificial Sequence
  amplification PCR miR397a mutagenic oligonucleotide primer miR397a-MF
tcgttcagagccgcattaatgtaatttcgttttgtttttcattgttaatgga

SEQ ID NO: 15 - DNA, 29 bp, Artificial Sequence
  amplification RT-PCR LAC cloning oligonucleotide primer LAC-F
gctctagagcatggggtctcatatggttt

SEQ ID NO: 16 - DNA, 24 bp, Artificial Sequence
  amplification RT-PCR LAC cloning oligonucleotide primer LAC-R
ggggtaccccttagcacaagggaa

SEQ ID NO: 17 - DNA, 24 bp, Artificial Sequence
  amplification RT-PCR CKB3 cloning oligonucleotide primer CKB3
gctctagagcatgtacaaggaacg

SEQ ID NO: 18 - DNA, 26 bp, Artificial Sequence
  amplification RT-PCR CKB3 cloning oligonucleotide primer CKB3
ggggtacccctcatggtttgtgtacc

SEQ ID NO: 19 - DNA, 44 bp, Artificial Sequence
  amplification PCR LAC mutagenic oligonucleotide primer LAC-MF
ttaacgcggcgctgaacgaagaactctttttcaaagtcgccggc

SEQ ID NO: 20 - DNA, 37 bp, Artificial Sequence
  amplification PCR LAC mutagenic oligonucleotide primer LAC-MR
tcgttcagcgccgcgttaactagtcgtagcagatagg

SEQ ID NO: 21 - DNA, 49 bp, Artificial Sequence
  amplification PCR CKB3 mutagenic oligonucleotide primer CKB3-MF
attaatgacgcgctgaatcaagaaactagagaaatcttcaacttccacc

SEQ ID NO: 22 - DNA, 40 bp, Artificial Sequence
  amplification PCR CKB3 mutagenic oligonucleotide primer CKB3-MR
gattcagcgcgtcattaattcgtttccgatcaatagctcc

SEQ ID NO: 23 - DNA, 32 bp, Artificial Sequence
  amplification RT-PCR miR397a promoter cloning oligonucleotide primer miR397a-GUS.F
gctctagagcttgcgcttcgtaccggtgagcg

SEQ ID NO: 24 - DNA, 32 bp, Artificial Sequence
  amplification RT-PCR miR397a promoter cloning oligonucleotide primer miR397a-GUS.R
cgggatcccgccaggaaaaaatatcctcatgc

SEQ ID NO: 25 - DNA, 32 bp, Artificial Sequence
  amplification RT-PCR miR397b promoter cloning oligonucleotide primer miR397b-GUS.F
gctctagagctcaatgatgttcattcaaaccc

SEQ ID NO: 26 - DNA, 32 bp, Artificial Sequence
  amplification RT-PCR miR397b promoter cloning oligonucleotide primer miR397a-GUS.R
cgggatcccgtttggaagttttgggtttctcc

SEQ ID NO: 27 - DNA, 28 bp, Artificial Sequence
  amplification RT-PCR LAC promoter cloning oligonucleotide primer LAC-GUS.F
aaattacaaaaccaagagatccacgacg

SEQ ID NO: 28 - DNA, 36 bp, Artificial Sequence
  amplification RT-PCR LAC promoter cloning oligonucleotide primer LAC-GUS.R
cgggatccctccctctctatctttctcttctctctc

SEQ ID NO: 29 - DNA, 31 bp, Artificial Sequence
  amplification RT-PCR CKB3 promoter cloning oligonucleotide primer CKB3-GUS.F
cgggtcgacataaaacaaaacatcgaatccg

SEQ ID NO: 30 - DNA, 32 bp, Artificial Sequence
  amplification RT-PCR CKB3 promoter cloning oligonucleotide primer CKB3-GUS.R
cgggatccattcgattccttctccaaaaagac

SEQ ID NO: 31 - DNA, 24 bp, Artificial Sequence
  RNA ligase-mediated 5′ RACE CKB3 gene-specific oligonucleotide primer CKB3-RACE1
accacaaagattgaaatcatcttg

SEQ ID NO: 32 - DNA, 28 bp, Artificial Sequence
  RNA ligase-mediated 5′ RACE CKB3 gene-specific oligonucleotide primer CKB3-RACE2
aaattacaaaaccaagagatccacgacg